(19) United States
(12) Patent Application Publication
(10) Pub. No.: US 2014/0163322 A1
(43) Pub. Date: Jun. 12, 2014

(54) SPECULUM COVER AND ASSEMBLY

(71) Applicant: Navroze S. Mehta, Boca Raton, FL (US)

(72) Inventor: Navroze S. Mehta, Boca Raton, FL (US)

(21) Appl. No.: 13/960,210

(22) Filed: Aug. 6, 2013

Related U.S. Application Data
(60) Provisional application No. 61/680,859, filed on Aug. 8, 2012.
(60) Provisional application No. 61/751,031, filed on Jan. 10, 2013.
(60) Provisional application No. 61/817,922, filed on May 1, 2013.

(51) Int. Cl. A61B 1/00; A61B 17/02; A61B 1/32

(52) U.S. Cl. CPC A61B 1/00135; A61B 1/32; A61B 17/02
USPC 600/203

(57) ABSTRACT

A disposable multi-planar speculum including a perforated flexible membrane adapted for examination and biopsy.

### CROSS REFERENCE TO RELATED APPLICATION

The present application is a United States Utility application conversion of several United States Provisional Applications namely: Multi-Planar Speculum, Application No. 61/680,859, filed Aug. 8, 2012; Multi-Planar Speculum, Application No. 61/751,031, filed Jan. 10, 2013; and Speculum Cover and Assembly, Application No. 61/817,922, filed May 1, 2013.

### BACKGROUND

Vaginal speculums are known from U.S. Pat. No. 6,432,048 among others. In the prior art the use of separate disposable sheath like membrane are known for use to facilitate vaginal examination with a speculum. These prior art devices suffer from several defects. Relative motion of the membrane or sheath with respect to the speculum as well as membrane retention are issues with these conventional prior art products. For this reason there is a continuing need to develop easily used devices to assist in gynecologic examination.

### SUMMARY OF THE INVENTION

Two embodiments of the invention are presented in this disclosure. In one embodiment the speculum is covered in part with a tubular sheath. In another embodiment the sheath is covered with sheet of material. The speculum device is similar in each embodiment. In each case the speculum device includes an upper blade and a lower blade that can move apart in a posterior/anterior motion. In one embodiment an integral membrane is deployed as the speculum opens. The membrane provides lateral retraction of tissues and is a very useful aid to examination. The membrane is thin and flexible and it is preferably manufactured from urethane and it is preferably adhered to the blades with an adhesive. The membrane has a series of spaced apertures along the lateral aspects that allow both visualization of tissue as well as access through the apertures for biopsy.

A first version of the membrane is fabricated from a tubular polymer structure and the second version is fabricated from a polymer sheet like material.

It is intended that the entire tool be a single use disposable instrument delivered in conventional packaging. However in a second embodiment it is preferred to apply a disposable cover sheet or tubular cover member to cover a re-usable, re-sterilizable speculum. After use, the cover material is removed from the speculum and it is discarded and the speculum re-sterilized.

### BRIEF DESCRIPTION OF THE DRAWINGS

Throughout the drawings identical reference numerals refer to identical structure wherein:

FIG. 1 is a view of the first embodiment speculum in the undeployed state;

FIG. 2 is a view of the first embodiment speculum in the deployed state;

FIG. 3 is a view in isolation of a tubular sheath element;

FIG. 4A is a perspective explosion view of the components of the second sheet like embodiment disposable speculum cover;

FIG. 4B is a perspective explosion view of the components of the second sheet like embodiment disposable speculum cover; and,

FIG. 5 is a perspective view of the components seen in FIG. A assembled into the disposable speculum cover.

### DETAILED DESCRIPTION

### Speculum Features

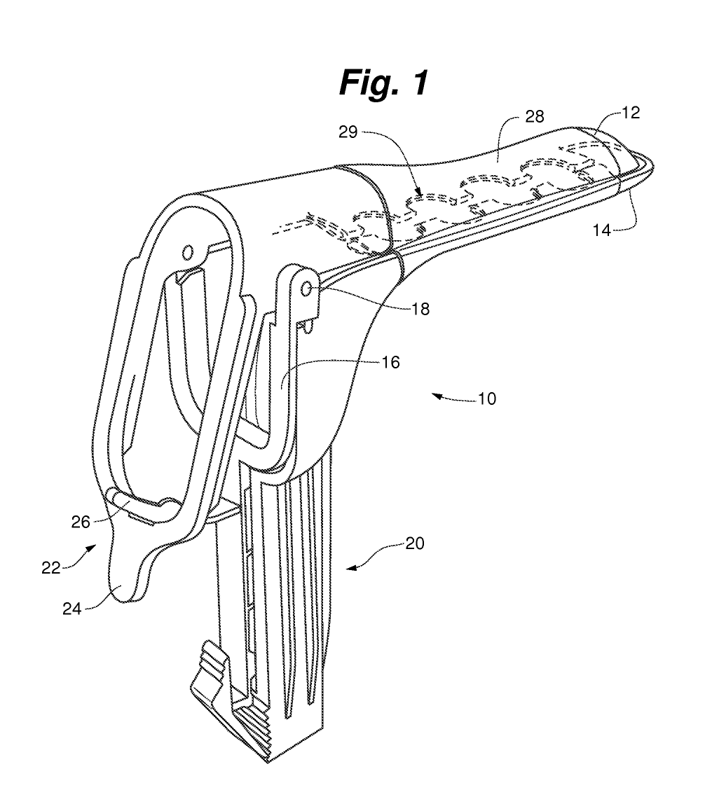

In a first embodiment as seen in FIG. 1 the speculum 10 is preferably made from a plastic material such as transparent polycarbonate, and it forms a single use disposable medical product. The speculum includes an upper blade 12 and a lower blade 14. Preferably the lower blade is formed with an anterior yoke 16 that carries the upper blade 12 on a pivoted axel 18. The axis of the pivot is perpendicular to a handle segment 20 of the speculum. A thumb actuated ratchet assembly 22, comprising a thumb lever 24 and a toothed sector 26 allow the physician to set the angle between the upper blade 12 and the lower blade 14. With the angle set to zero as seen in the figure a portion of the membrane 28 is trapped within the speculum. In the figure the trapped segment of the membrane is shown in dotted outline and indicated by reference numeral 29. In this predeployment configuration the membrane is protected during insertion.

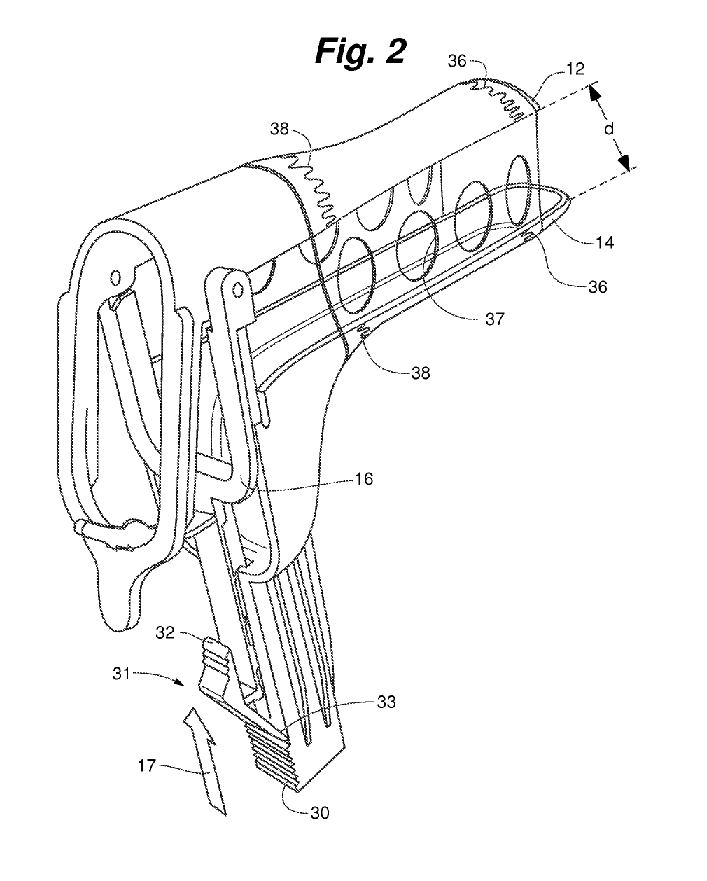

As seen in FIG. 2 the yoke 16 may translate as indicated by motion arrow 17 along the handle, thus spacing the upper and lower blades apart a distance labeled “d” in the figure. A ratchet system 31 comprising a toothed flat surface 30 and a complimentary pawl element 33 coupled to a manually operated lever 32.

In use the physician actuates both ratchet systems 31 and 22 to fit the speculum for the examination. Once deployed the membrane 28 stretches to conform to the positions of the blades 12 and 14.

### Membrane Features

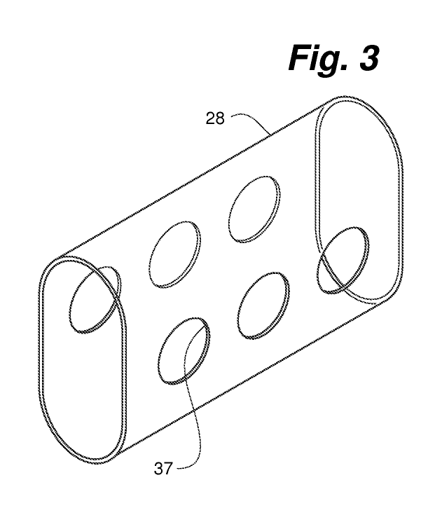

FIG. 3 shows the membrane structure in isolation with an interior and exterior. In general the membrane structure is tubular and bonded to sections of the upper and lower blades. The fixation features allow the membrane to stretch to accommodate the position of the blades without detaching or tearing. In the prior art under some conditions of use the membrane would separate from blades.

In general it is preferred to fabricate the membrane from a uniform and thin layer of polyurethane that is transparent. A series of holes, windows or apertures are located bilaterally along the edges of the opposed blades. Window 37 typifies an aperture bilaterally located across from a complimentary aperture in the lateral position.

### Fabrication Features

The preferred membrane is formed from polyurethane as a sheath or tube. Several holes are cut into the lateral aspects of the membrane to facilitate observation of tissue and to permit biopsy. The preferred membrane is attached to speculum to form an integrated device adapted for a single use. The attachment points cooperate to facilitate deployment and use of the membrane. In general the speculum is assembled and the tubular sheath of the membrane is positioned over the blades. Typically the blades are parallel and spaced a characteristic distance apart “d”. The apertures in the membrane typified by aperture 34 are located so that they do not contact the blade surfaces. Next an adhesive is placed at separated locations along the blade membrane interface. In the figures glue lines are identified by reference numerals 36 and 38. Glue line 36 is positioned on the distal tip of the upper blade 12. The glue is interrupted by the edge of the blade and is then continued on to the lower blade 14 and identified with the same reference numerals. In a similar fashion the glue line 38 is on the forward or the proximal portion of the speculum. These glue lines are nearly invisible in the final product and preferably the adhesive used is a UV cure medical grade adhesive for plastic such as Loctite 3971. Once cured the speculum is “let down” and the membrane folded over the two blades into the interior as seen in FIG. 1. Thereafter the product is packaged for use.

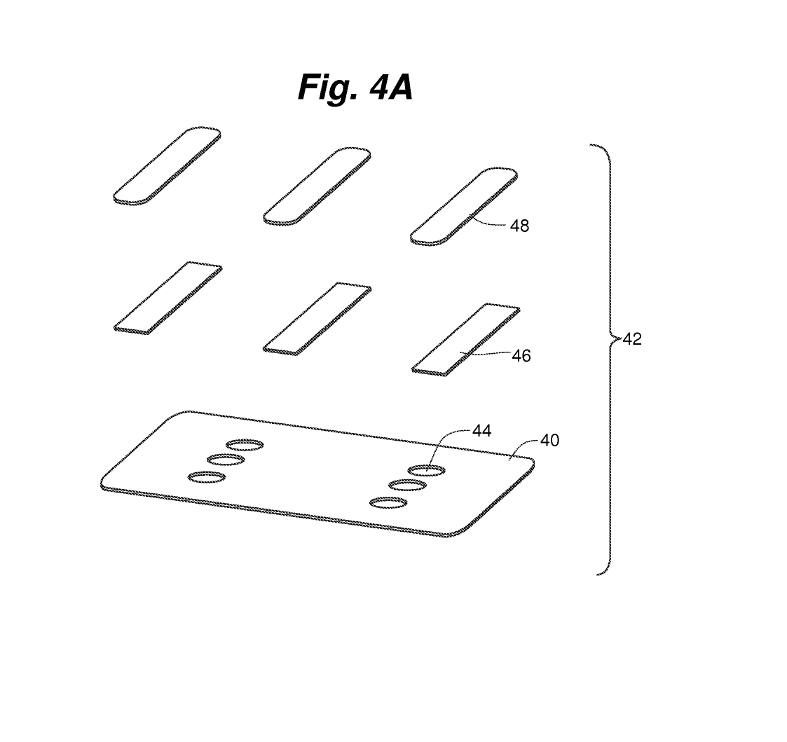

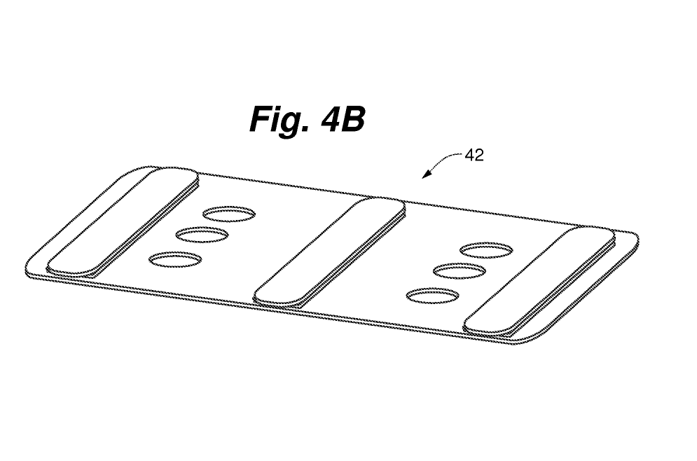

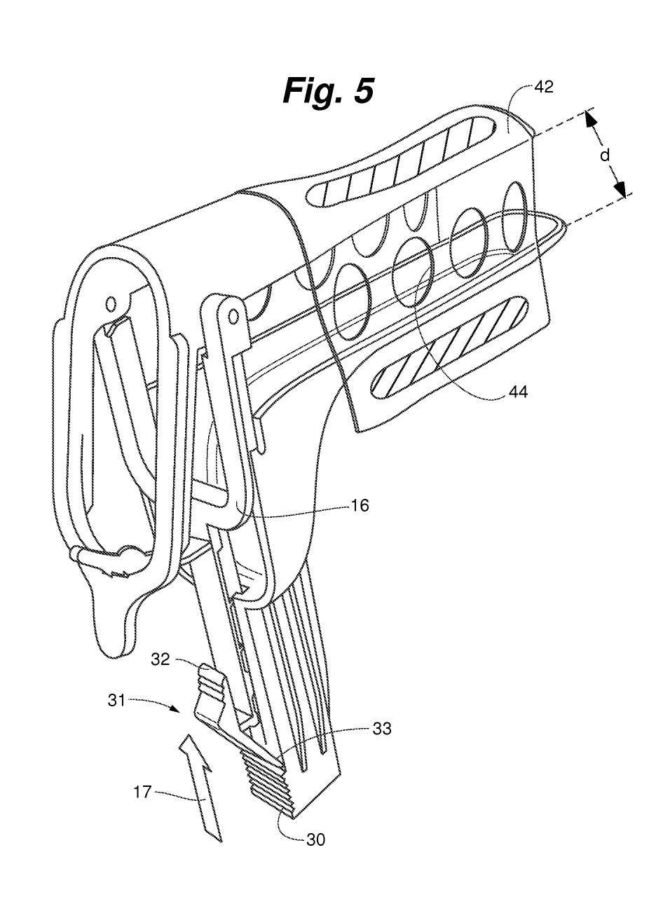

In a second embodiment as seen in FIG. 4A and FIG. 4B and FIG. 5 a unitary planar sheet of polymer 40 is provided for application to the speculum body seen in FIG. 4 as 42. The planar liner sheet 40 forms a disposable single use speculum cover. The disposable speculum cover has several apertures or windows arrayed in its surface typified by liner aperture 44. The aperture array is sized to align with the sides of the speculum so that they are stretched into position as the speculum is deployed.

In between the aperture array is a set of adhesive areas that are formed by applying adhesive strips to the liner sheet 40. The adhesive strips are typified by strip 46. In the explosion view the strip are shown above the liner locations where they are applied. Backer sheets typified by backer sheet 48 are applied to cover each individual adhesive strip.

In use the physician will remove one backer sheet (typically the center sheet) and apply the liner to the upper blade of the speculum. Next the approximate opening will be set by the physician and the remaining backer sheet will be removed and the remaining adhesive areas applied to the lower jaw of the speculum.

This completes assembly and the speculum is ready for use.

What is claimed is:

1. A speculum comprising:
a pair of blades consisting of a first blade and a second blade;
said first blade and said second blade movable in an arc path about a pivot axis;
said first blade and said second blade movable in along a linear path between an open position and a closed position;
a flexible tubular membrane surrounded said first and second blades and coupled to the distal portion of each blade with a band of glue;
said flexible tubular membrane surrounded said first and second blades and coupled to the proximal portion of each blade with a band of glue.

2. The speculum of claim 1 further comprising:
a first ratchet assembly for positioning said first and second blades along said arc path;
a second ratchet assembly for positioning said first and second blades along said linear path.

3. The speculum of claim 2 further comprising:
said membrane having a series of windows or holes along its length that are opened when said first and second blades move along said linear path.

4. A speculum comprising:
a handle having a length;
a pair of blades consisting of a first blade and a second blade;
said first blade and said second blade movable in an arc path about a pivot axis;
a movable yoke adapted to receive said pivot axis and thereby define said arc path;
said movable yoke adapted to move along a linear path defined along the length of said handle thereby moving said first and second blades between an open position and a closed position;
a flexible tubular membrane fabricated from a uniform thin layer of polyurethane surrounded said first and second blades and coupled to the distal portion of each blade with a band of UV curable glue;
said flexible tubular membrane surrounded said first and second blades and coupled to the proximal portion of each blade with a band of UV curable glue.
a first ratchet assembly attached to said yoke for positioning said first and second blades along said arc path;
a second ratchet assembly attached to said handle for positioning said first and second blades along said linear path.
said membrane having a series of pairs bilaterally opposed and positioned windows along its length that are opened when said first and second blades move along said linear path.

5. A speculum cover comprising:
a planar polymer sheet;
an array of apertures perforating said polymer sheet;
at least three adhesive patches located on said polymer sheet spaced apart from said apertures;
a set of backer sheets coving said adhesive patches.

6. A speculum cover comprising:
a tubular polymer sheet;
an array of apertures perforating said polymer sheet;
at least three adhesive areas located on said polymer sheet spaced apart from said apertures;
a set of backer sheets coving said adhesive patches.
